(19) United States
(12) Patent Application Publication
(10) Pub. No.: US 2006/0041016 A1
(43) Pub. Date: Feb. 23, 2006

(54) Composition and method for the treatment of psoriasis

(76) Inventors: Johan Stjernschantz, Mariehamn (FI); Bahram Resul, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 11/204,491

(22) Filed: Aug. 16, 2005

(30) Foreign Application Priority Data
Aug. 17, 2004 (SE) 0402029-3

(51) Int. Cl. A61K 31/557

(52) U.S. Cl. 514/530; 514/573; 514/613

(57) ABSTRACT

The use of a prostaglandin A₂ derivative, and prodrugs of the compound, for the manufacture of a medicament for the treatment and/or alleviation of psoriasis is presented, as well as a method of treatment, involving the topical application of such prostaglandins. Compositions containing a therapeutically active, and physiologically acceptable amount of the above compound or derivatives thereof, as such or in the form of a prodrug, in a suitable vehicle are also described. Importantly, the derivatives can be applied in therapeutically active amounts without or with only minimal side effects, such as hyperemia, irritation or pain.

### FIELD OF THE INVENTION

The present invention relates to a composition and method for the treatment and/or alleviation of psoriasis, the use of a prostaglandin A₂ (PGA₂) derivative for this purpose, as well as the use of said derivative and pharmaceutically acceptable prodrugs thereof, for the manufacture of a medicament for the treatment and/or alleviation of psoriasis.

### BACKGROUND OF THE INVENTION

Psoriasis is a common skin disorder affecting 1-2% of the population in Europe and USA. The disease is inherited and usually debuts at the age of 10-40 years, but may become manifest at any age. There are many subtypes of psoriasis, but in the most common form of the disease hyperkeratotic pink lesions covered by adherent silver-white scales can be found in afflicted patients. Not infrequently the lesions are localized to the elbows, knees, the gluteal regions, and the scalp. It is generally regarded that physical contact, such as pressure and rubbing of the skin, may provoke the disease.

The underlying mechanism of psoriasis is believed to be inflammation leading to increased proliferation of cells in the epidermis, primarily keratinocytes. Thus, the epidermis becomes thick, and hyperkeratotic. Inflammatory mediators such as tumour necrosis factor alpha (TNF_{α}) are believed to be important components in the pathogenesis of the disease. Psoriasis may also become generalized over the whole body and the disease may lead to arthritis, typically in the fingers. The course of the disease may fluctuate, but complete and permanent remission is uncommon.

Psoriasis is usually treated with different medications. In simple cases keratolytics, lubricants and topical corticosteroids are employed. Salicylic acid, tar-based medicines, and anthralins and vitamin D preparations are also used. In more wide-spread psoriasis PUVA-treatment may be employed. This treatment is based on systemic or local administration of psoralens, e.g. 8-methoxypsoralen, in combination with irradiation of the skin with ultraviolet light (UVA). This treatment modality is effective, but may predispose to skin cancer. Antimitotics, such as methotrexate, have also been employed in severe cases of psoriasis. More recently, antibodies, or fusion proteins to T-lymphocytes have been found effective in severe cases of psoriasis, but a disadvantage is that the medication has to be given by intravenous injection. A systemic administration always increases the risk of unwanted side effects. Also retinoids may be used for the treatment of psoriasis. Although currently many treatment modalities for psoriasis exist, there is a definite need for more effective medications with less side effects.

### PRIOR ART

Several patents deal with the use of prostaglandins, including prostaglandin A, for the treatment of psoriasis, e.g. DE 2460285, which discloses specific trans-delta-2 prostaglandin derivatives of type A, E and F, as well as methods for their synthesis and pharmaceutical use. These trans-delta-2 prostaglandin derivatives are suggested for the treatment of a variety of autoimmune diseases, including psoriasis.

U.S. Pat. No. 4,254,145 discloses a large variety of prostaglandins, including PGA type prostaglandins, claimed to be useful for the treatment of psoriasis. The presumed mechanism of action was described to be increased peripheral blood circulation.

In a more recent patent patent, U.S. Pat. No. 6,031,001 (Stjernschantz and Resul), prostaglandins of the A and J type are disclosed as being useful for the treatment of psoriasis based on their antiproliferative effect on human keratinocytes.

Considering the general disclosures of the prior art, it still remains desirable to develop specific prostaglandins for the treatment of psoriasis.

### SUMMARY OF THE INVENTION

The present invention makes available a composition for topical application, said composition containing a therapeutically active and physiologically acceptable amount of 17-phenyl-18,19,20-trinor-PGA₂ or a derivative thereof for the treatment and/or alleviation of psoriasis.

Another embodiment of the invention concerns the use of 17-phenyl-18,19,20-trinor-PGA₂ or a derivative thereof for the manufacture of a medicament containing a therapeutically active and physiologically acceptable amount of said prostaglandin for the treatment and/or alleviation of psoriasis.

A third embodiment concerns a method for the treatment and/or alleviation of psoriasis in a human patient, wherein a therapeutically active and physiologically acceptable amount of a prostaglandin is applied topically to a psoriatic skin lesion on the skin of said patient, wherein said prostaglandin is 17-phenyl-18,19,20-trinor-PGA₂ or a derivative or prodrug thereof.

In all three embodiments, it is of great significance that both antiproliferative and apoptotic effect is achieved in the absence of unwanted side-effects such as hyperemia, irritation or pain.

### SHORT DESCRIPTION OF THE DRAWINGS

The invention will be described in closer detail in the following description, examples, and attached drawings, in which

FIG. 1 shows the effect of PGA₂ on normal human epithelial keratinocytes in a spectrophotometric analysis. The compound was added in the following concentrations: A1=10⁻⁴M; A2=3×10⁻⁵M; A3=10⁻⁵M; A4=3×10⁻⁶M; A5=10⁻⁶M; and A6=10⁻⁷M.

FIG. 2 shows the effect of BR277 (16-phenoxy-(3-trifluormethyl)-17,18,19,20-tetranor-PGA₂). The compound was added in the following concentrations: B1=10⁻⁴M; B2=3×10⁻⁵M; B3=10⁻⁵M; B4=3×10⁻⁶M; B5=10⁻⁶M; and B6=10⁻⁷M.

FIG. 3 shows the effect of BR278 (13,14-dihydro-17-phenyl-18,19,20-trinor-PGA₂). The compound was added in the following concentrations: C1=10⁻⁴M; C2=3×10⁻⁵M; C3=10⁻⁵M; C4=3×10⁻⁶M; C5=10⁻⁶M; and C6=10⁻⁷M.

FIG. 4 shows the effect of BR280 (17-phenyl-18,19,20-trinor-PGA₂). The compound was added in the following concentrations: D1=10⁻⁴M; D2=3×10⁻⁵M; D3=10⁻⁵M; and D4=3×10⁻⁶M; D5=10⁻⁶M; D6=10⁻⁷M

### DESCRIPTION

The term “prodrug” is intended to define any substance that gives rise to a pharmacologically active metabolite, although not itself active or only partially active (i.e. precursor of the pharmacologically active compound).

The terms “derivative” and “functionally equivalent derivative” are intended to encompass any chemical compound or substance, derived or obtained from another substance and exhibiting the same, similar or improved properties, e.g. efficacy, stability, tolerability etc, as the parent compound. A functionally equivalent derivative of a drug can be designed by retaining the general structure of the drug molecule, but adding or removing atoms or moieties that do not influence the therapeutic function of the drug.

Prostaglandins are fatty acids derived from unsaturated fatty acid precursors through metabolic steps involving oxygenation. The most important and common precursor is arachidonic acid, or eicosatetraenoic acid, and prostaglandins derived from this precursor are of the 2-series. The general structure of prostaglandins comprise a cyclopentane ring to which two carbon chains attach, the upper usually being called the alpha chain, and the lower the omega chain. The alpha chain is a 7 carbon carboxy-terminated aliphatic chain whereas the omega chain is an 8 carbon methyl-terminated aliphatic chain. Depending on the number of double bonds in these chains subscripts of 1-3 are given. In prostaglandins of the 2 series the alpha chain carries a double bond in cis-configuration between carbons 5 and 6, and the omega chain carries a double bond in trans-configuration between carbons 13 and 14. The prostaglandins carry a hydroxyl group on carbon 15 in the omega chain, which is important for biologic activity.

The prostaglandins are furthermore classified in subgroups of A, B, C, D, E, F, and J depending on the structure and substituents of the cyclopentane ring. In prostaglandins of A type with a carbonyl group at carbon 9, a double bond exists between carbons 10 and 11, and the ring structure is accordingly called cyclopentenone.

The present invention concerns 17-phenyl-18,19,20-trinor-PGA₂, a specific derivative of PGA₂, and prodrugs and derivatives thereof. Prostaglandins of the A type may be formed from E type prostaglandins in acidic environment, and the present invention also relates to the specific PGA₂ analogue produced synthetically, or by biochemical means from the corresponding PGE₂ analogue. The chemical structure of 17-phenyl-18,19,20-trinor-PGA₂-isopropyl ester, a prodrug form of 17-phenyl-18,19,20-trinor-PGA₂, is depicted below (compound 1).

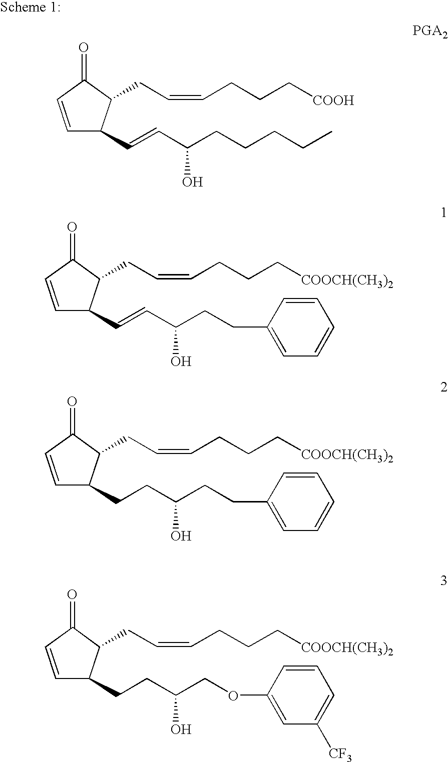

The present inventors have now found that one specific PGA₂ derivative, namely 17-phenyl-18,19,20-trinor-PGA₂, and prodrugs of this compound, appear exceptionally suitable for the treatment of psoriasis. This prostaglandin derivative is an omega chain ring substituted analogue of PGA₂, a previously not disclosed member of a group of prostaglandin analogues generally claimed to be useful for the treatment of psoriasis (WO 96/09055; U.S. Pat. No. 6,031,001).

The inventors have now showed that such derivatives of PGA₂ indeed all show an antiproliferative effect on human keratinocytes (the examples in the present specification comprise 17-phenyl-18,19,20-trinor-PGA₂, 13,14-dihydro-17-phenyl-18,19,20-trinor-PGA₂ and 16-phenoxy-(3-trifluormethyl)-17,18,19,20-tetranor-PGA₂).

However, among the compounds tested, 17-phenyl-18,19,20-trinor-PGA₂ exhibited superior properties. This compound in fact, surprisingly, exhibited higher antiproliferative effect than PGA₂ itself. In addition, 17-phenyl-18,19,20-trinor-PGA₂ caused apoptosis of human keratinocytes, a very beneficial effect in the prevention of hyperproliferation of keratinocytes in psoriasis.

Additionally, the inventors found that 17-phenyl-18,19,20-trinor-PGA₂ is an exceptionally advantageous compound, because in contrast to PGA₂ it exhibits no hyperemic effect or irritative effect whatsoever, when tested on the human eye, an excellent model of hyperemia/irritation. The hyperemia is based on increased diameter of the blood vessels causing the tissue to appear red, one of the typical and undesirable symptoms in psoriasis.

All prostaglandins tested so far by the inventors (including omega chain ring substituted prostaglandins, see U.S. Pat. No. 5,422,368) cause to some extent increased blood flow (increased blood vessel diameter). In addition, many prostaglandins also cause irritation or pain in the tissue to which they are applied. Notably, PGA₂ causes both these disadvantageous side-effects. 17-phenyl-18,19,20-trinor-PGA₂ however appears to be completely devoid of these side-effects while exhibiting higher antiproliferative effect than PGA₂.

With reference to the compounds disclosed in U.S. Pat. No. 5,422,368, also 13,14-dihydro-17-phenyl-18,19,20-trinor-PGA₂ when tested in the form of an isopropyl ester prodrug caused some hyperemia in the eye. The same compound was also tested for antiproliferative effect in the series of experiments performed for the current patent specification and was found to exert significantly less antiproliferative effect than 17-phenyl-18,19,20-trinor-PGA₂. Thus 17-phenyl-18,19,20-trinor is exceptionally suitable for topical treatment of psoriasis. It follows, naturally, that prodrugs of this specific compound e.g. carboxylic acid esters would be equally suitable for treatment of psoriasis.

In summary, the inventors have shown that 17-phenyl-18,19,20-trinor-PGA₂ exerts significantly higher antiproliferative effect and causes no hyperemia, irritation or pain. It is shown to be superior to comparable omega chain ring-substituted PGA₂ analogues, such as 13,14-dihydro-17-phenyl-18,19,20-trinor-PGA₂ and 16-phenoxy-(3-trifluormethyl)-17,18,19,20-tetranor-PGA₂. Importantly, the 17-phenyl-18,19,20-trinor-PGA₂ analogue furthermore was shown to cause keratinocytes to undergo apoptosis, programmed cell death, which contributes to the antiproliferative effect of the drug.

Thus, the present invention makes available a composition for the treatment and/or alleviation of psoriasis. Compositions comprising 17-phenyl-18,19,20-trinor-PGA₂ or functional derivatives thereof, including prodrugs thereof, e.g. carboxylic acid esters as well as amides, may be formulated as solution, gel, lotion, cream, ointment, shampoo, or other form topically acceptable for use on skin or hair (scalp).

The preferred compound, 17-phenyl-18,19,20-trinor PGA₂, functionally equivalent derivatives thereof, as well as prodrugs of this compound, should be used in a suitable vehicle for topical application on the skin. Suitable vehicles include aqueous vehicles, with or without solubilizers and stabilizers such as cyclodextrins, oils, ointments, creams, gels, micelle systems, nanoparticles and various slow release formulations. Suitable vehicles may or may not contain preservatives depending on whether they are intended for single or multiple use. Various preservatives that may be employed comprise e.g. benzalkonium chloride, chlorhexidine, thiomersal, parabenzoic acid, alcohols, and other agents with satisfactory antimicrobial effect.

To prepare a combination composition for synergetic effects, a cosmetic, pharmaceutical or other topical agent is incorporated into any one of the above compositions by dissolving or mixing the agent into the formulation. Examples of such cosmetic, pharmaceutical or other agents include, but are not limited to, emollients, moisturisers, essential fatty acids, anti inflammatory drugs, sunscreen agents, perfumes, colouring agents etc.

Other forms of compositions for topical delivery of 17-phenyl-18,19,20-trinor-PGA₂ compound or compounds, are readily prepared or formulated by those skilled in the art.

An example of one formulation containing the preferred compound is presented in Table 1.

TABLE 1
Formulation containing the preferred prostaglandin analogue

| 17-phenyl-PGA₂-IE* | 0.1 mg; 0.3 mg; 1 mg/gram |
|---|---|
| Unguentum macrogolum 300 et 1540 | 49.2% |
| (0.5:0.5) | |
| Glycerolum (max 0.5% aq) | 49.2% |
| Ethanolum | 1.6% |

*17-phenyl-18,19,20-trinor-PGA₂-isopropyl ester

Further, the present invention makes available a method for the treatment and/or alleviation of psoriasis. Accordingly, 17-phenyl-18,19,20-trinor-PGA₂ or functional derivatives thereof, used as such or in the form of a prodrug, e.g. as isopropyl ester, is applied topically on the affected skin for different periods of time, once or several times daily, for treatment of the psoriatic lesions. Such treatment may take only a few weeks or may go on for longer time periods depending on the clinical situation. The recommended dose to be used is in the range of 0.01 to 100 μg per application. On an area of 1 dm² typically a dose of 1-100 μg per application is employed. The medication can be instilled once or several times daily depending on the clinical situation and the dosage form. When the psoriatic lesion has regressed, treatment may continue intermittently, or may be terminated.

The invention also relates to the use of 17-phenyl-18,19,20-trinor-PGA₂, and/or its prodrugs as defined above for the preparation of a composition for treatment of psoriasis. The doses, concentrations, compositions and their components given above are applicable also to this use, mutatis mutandis.

The invention is illustrated with the following non-limiting examples.

### EXAMPLES

The prostaglandin analogues used for the exemplification of the invention were synthesized as set forth in the paragraphs beneath. The compounds 1, 2 and 3 were prepared according to a published method (Resul et al. (1993), Phenyl-susbstituted prostaglandins: potent and selective antiglaucoma agents. J Med Chem 36: 243-248), slightly modified as will be evident from the account of the synthesis given below.

General Methods for Synthesis of PGA₂ Analogues

Method A Preparation of Compound 1 (Scheme II)

Compound 1 was synthesized from Corey's lactone 4. The primary alcohol of lactone 4 was oxidized to aldehyde 5 with DCC, DMSO and H₃PO₄ in DME. The crude aldehyde 5 was reacted with dimethyl (2-oxo-4-phenylbutyl) phosphonate in the presence of lithium chloride and diisopropyl ethyl amine to give 6 in 70% yield. Stereoselective reduction of the enone 6 with S-Alphine hydride in THF at −78° C. provided 70-75% of the (15S)-7 isomer over the (15R)-7 isomer. Alternatively, the reduction could be performed with NaBH₄.CeCl₄(H₂O)₇ in MeOH/CH₂Cl₂ with lower selectivity. The epimers were readily separated by column chromatography on silica gel using tolene:ethyl acetate 2:1 as eluent to give pure (15-S)-7. Removal of the p-phenylbenzoyl (PPB) protective group with K₂CO₃ in MeOH afforded an 80% yield of the diol 8. The product 8 was purified by column chromatography on silica gel using ethyl acetate as eluent. The free hydroxyl groups of 8 were protected with tetrahydropyranyl (THP) groups to give 9 in quantitative yield. Compound 9 was treated with diisobutylaluminium hydride (DIBAL-H) in dry THF to give the lactol 10. Product 10 underwent Wittig reaction with 4-carboxybutyl-triphenylphosphonium bromide, t-BuOK in dry THF affording the acid 11. This was further reacted without isolation with isopropyl iodide in the presence of DBU in acetone to give the corresponding ester 12, which was isolated by column chromatography on silica gel using ethyl acetate:hexane 1:1. Compound 12 was oxidized with pyridinium chlorchromate treated with aluminium oxide (PCC/Al₂O₃) in dichloromethane to give the compound 13. The protecting groups 11, 15-bistetrahydro-pyranyloxy groups were removed by addition of 1N HCl in acetonitrile. The reaction mixture was stirred at room temperature for about 3 hrs to give 14. This was further reacted without isolation with addition of excess 1N HCl. The mixture was stirred for additional 40 hrs to accomplish quantitative conversion to the corresponding PGA₂ ester analogue (1).

Method B Preparation of Compound 2 (Scheme III)

Compound 2 was synthesized as outlined in Scheme III. The trans allylic double bond of compound 7 (Scheme I) was reduced under hydrogen atmosphere using Pd—C as a catalyst in the presence of sodium nitrite to give 15. Compound 15 was isolated and reacted subsequently following method A described above to give the desired product 2.

Method C Preparation of Compound 3 (Scheme IV)

Compound 3 was synthesized as outlined in Scheme IV utilizing the procedure described for synthesis of compound 2 (Method B) with minor modifications. The aldehyde 5 was reacted with the acyl phosphonate dimethyl(2-oxo-3-phenoxypropyl)phosphonate 23 (Scheme IV) to afford the enone 24. This was isolated and reacted subsequently following method B to give the desired product 3. 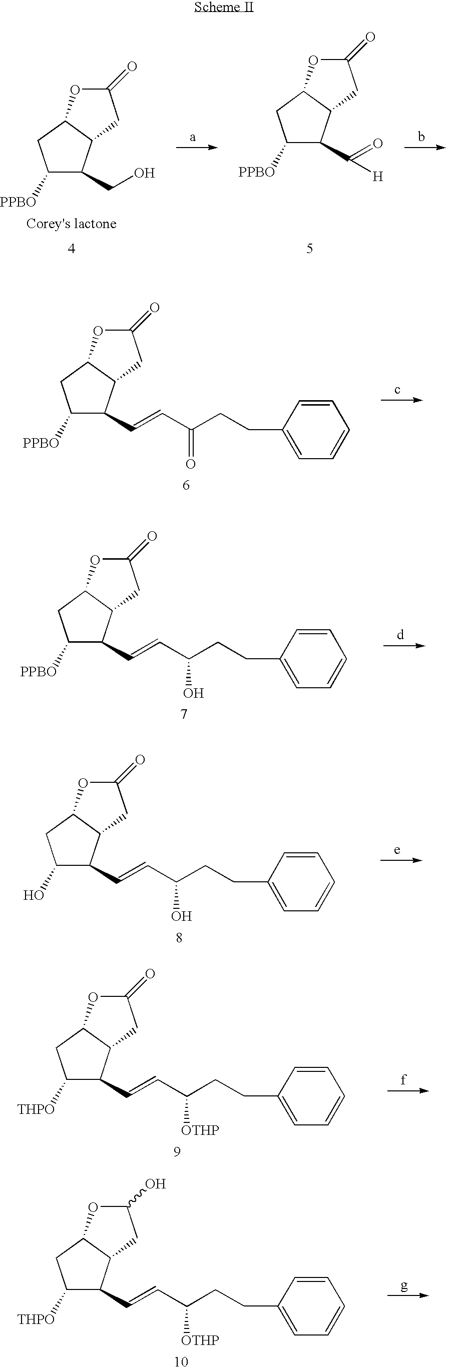 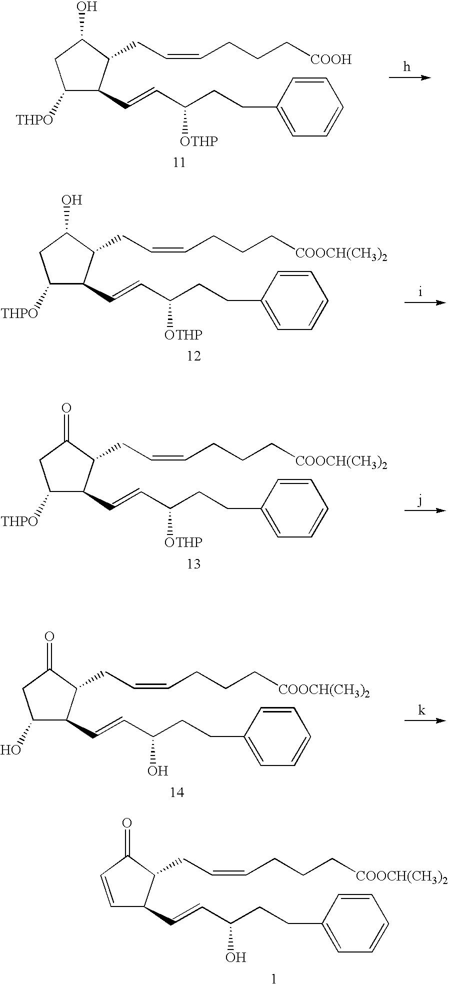
Reagents: a. DCC, DMSO, H₃PO₄/DME; b. LiCl, (CH₃O)₂POCH₂—CO—(CH₂)₂C₆H₅, DIPEA/CH₃CN-15° C.; c. S-Alpin hydride/THF; d. K₂CO₃/Methanol; e. DHP, PPTS/CH₂Cl₂; f. DIBAL-H/THF, −78° C.; g. 4-carboxybutyl triphenylphosphonium bromide, KOtBu/THF; h. iPrI, DBU/CH₃CN; i. PCC, Al₂O₃/CH₂Cl₂; j, k. 1N HCl/CH₃CN 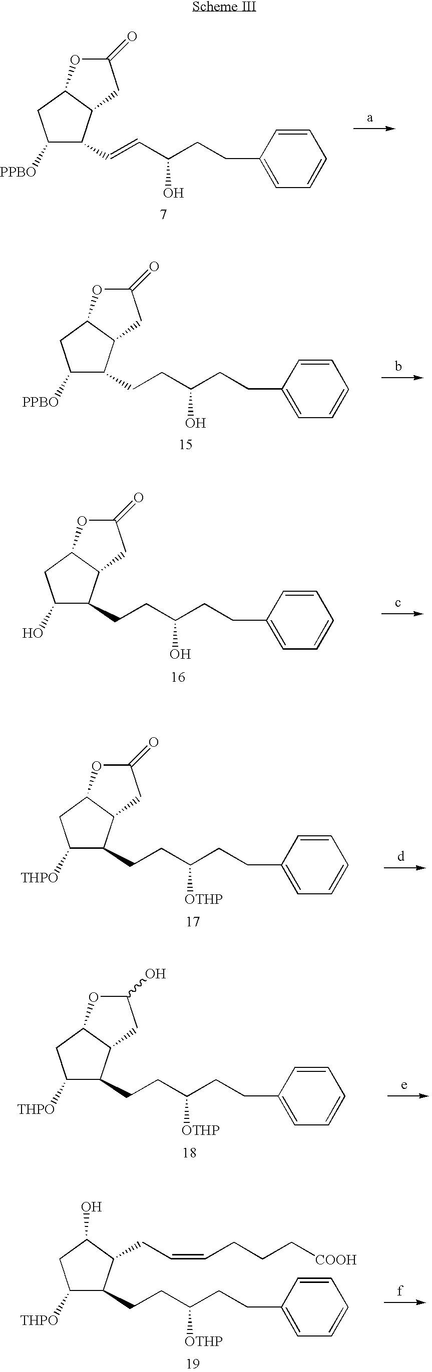 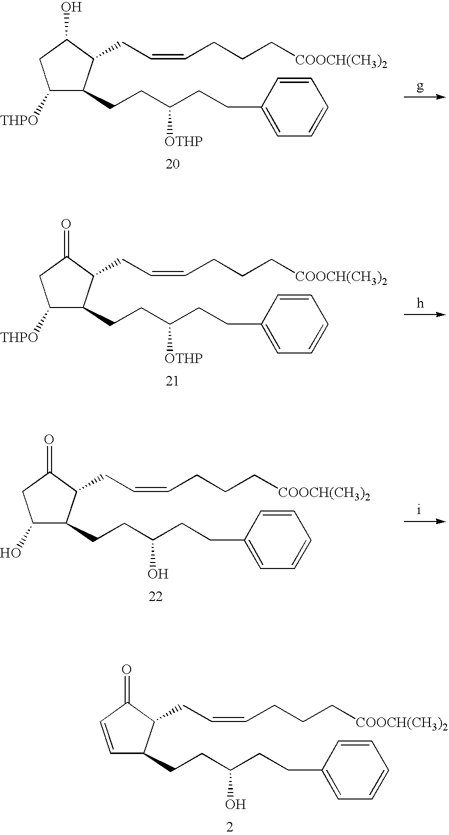
Reagents: a. Pd—C/H₂, NaNO₂, EtOH; b. K₂CO₃/Methanol; c. DHP, PPTS/CH₂Cl₂; d. DIBAL-H/THF; e. 4-carboxybutyl triphenylphosphonium bromide, KOtBu/THF; f. iPrI, DBU/CH₃CN; g. PCC, Al₂O₃/CH₂Cl₂; h, i. 1N HCl/CH₃CN 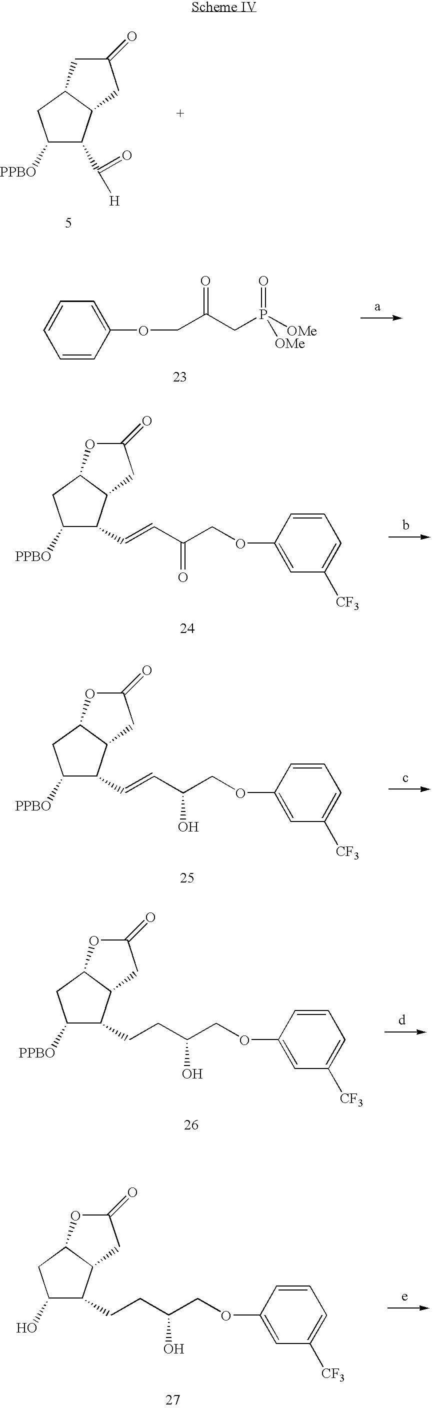 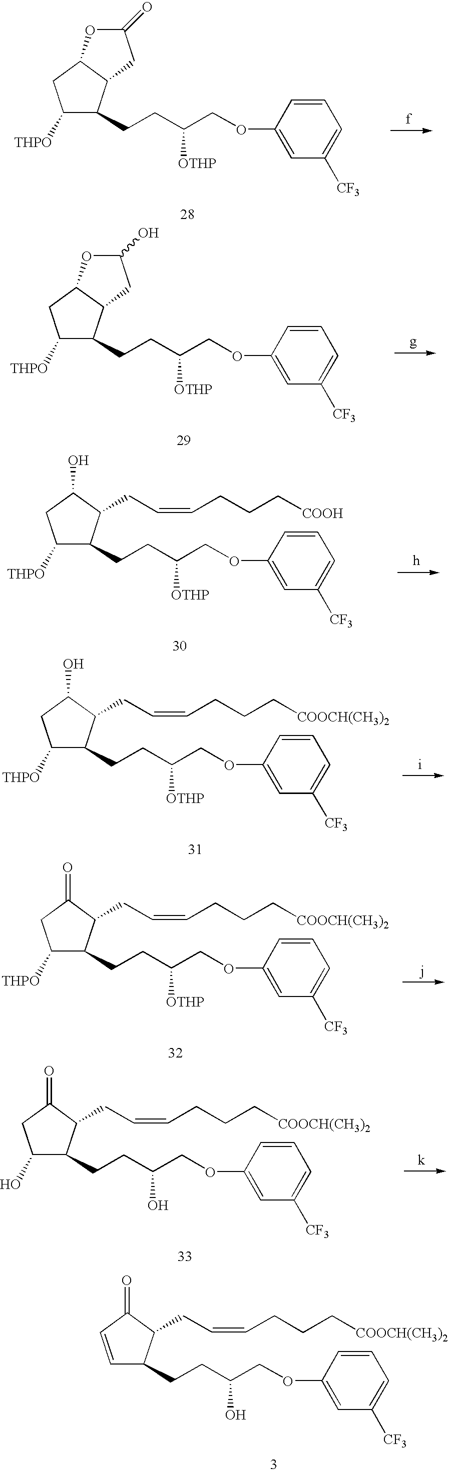
Reagents: a. NaH, DME; b. S-Alpin hydride/THF; c. Pd—C/H₂, NaNO₂, EtOH; d. K₂CO₃/Methanol; e. DHP, PPTS/CH₂Cl₂; f. DIBAL-H/THF; g. 4-carboxybutyl triphenylphosphonium bromide, KOtBu/THF; h. iPrI, DBU/CH₃CN; i. PCC, Al₂O₃/CH₂Cl₂; j, k. 1N HCl/CH₃CN
Preparation of PGA₂ Acids 1a, 2a and 3a (Scheme V)

In order to evaluate the in vitro activity of the PGA₂ analogues, the isopropyl ester prodrugs (Scheme I) were hydrolyzed with lipase type VII (Sigma) in phosphate buffer (pH7.4):acetone (1:0.2). The mixture was stirred magnetically at room temperature for 3 days (TLC monitoring, ethyl acetate). The reaction mixture was washed with ether. The water layer was extracted with ethyl acetate. The organic phase was separated and dried on magnesium sulphate and filtered. The solvent was removed in vacuo to give 1a, 2a and 3a as oils under the code names of BR280, BR278 and BR 277, respectively. 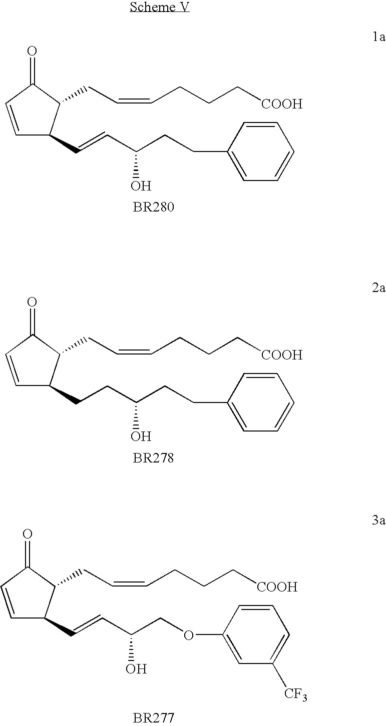
Antiproliferative Effect of Test Compounds

Normal human epithelial keratinocytes, and keratinocyte growth medium (KGM^{R}-2 Bulletkit; CC-3107 from Clonetics, USA) were purchased from Göteborgs Termometerfabrik AB (Västra Frölunda, Sweden). The cells were cultured at 37° C. in 5% CO₂ humidified air according to standard procedure. The cells used for the experiments were at the third passage. 48-well plates were used for the cultures. In each well about 3000 cells were seeded. For each concentration of prostaglandin analogues two wells were used. After completion of the incubation, the wells were washed twice with phosphate buffered saline (PBS). Thereafter the cells were fixed with 1% glutaraldehyde in PBS at +4° C. for 15 min. The wells were then again washed twice with PBS, and thereafter treated with 0.1% crystal violet solution (500 μl/well). The wells were then rinsed with distilled water for 15 sec, three times, and were left to dry completely. The absorbed colour was solubilized in 500 μl 2.5% sodiumdodecyl-sulphate (SDS) in distilled water for 3 hours. The absorbance of the fluid in the wells was determined using a microtiter well spectrophotometer (Multiscan RC V1.3-0) at 595 nm. The experiments were performed in duplicate (two wells). The incubation time was 7 days, and the incubation medium was changed after 3-4 days. The prostaglandin analogues were dissolved directly in the incubation medium. After appropriate incubation the density of cells in the wells was determined using the crystal violet method described above.

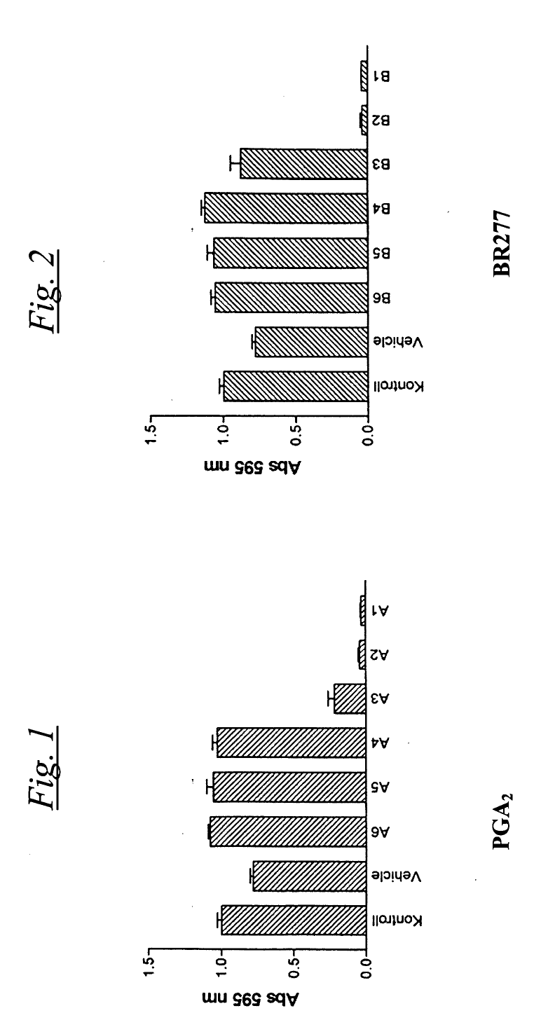

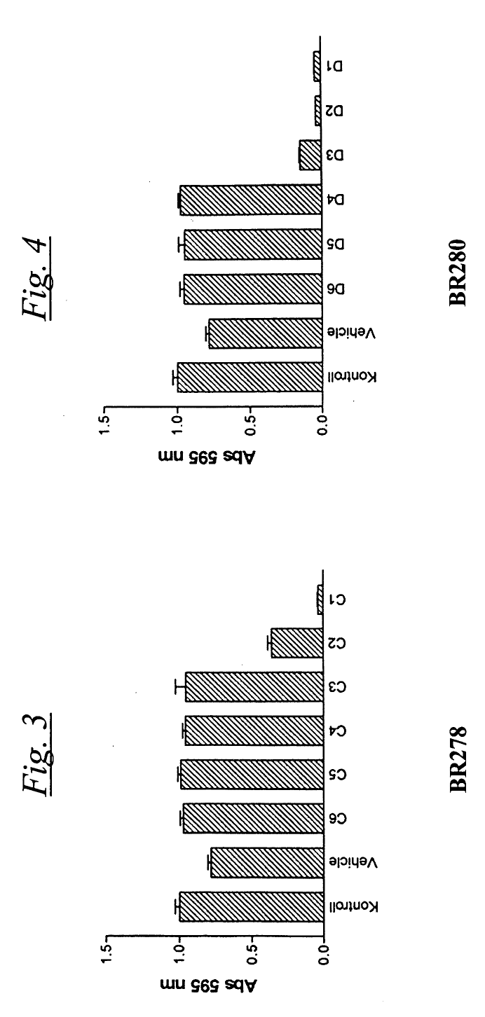

The results of the experiments are displayed in FIG. 1-4. BR280 (17-phenyl-18,19,20-trinor-PGA₂) appeared to be even potent than PGA₂, with an EC₅₀ value between 10⁻⁵M and 3×10⁻⁶M. BR277 was the next most potent of the PGA₂ analogues, BR278 exhibiting lowest potency. At concentrations of 10⁻⁶M and less none of the compounds had any effect. Neither PGA₂ nor any of the analogues tested were able to cause complete cell death even at the highest concentration tested. The maximum effect appeared to be around 95%. Thus, BR280 exhibited significantly better antiproliferative effect than the other two omega chain ring-substituted PGA₂ analogues, BR277 and BR278, and appeared to be even more potent than PGA₂.

Apoptotic Effect of 17-phenyl-18,19,20-trinor-PGA₂

In one set of experiments the expression of the pro-apoptotic BAX gene was demonstrated by immunocytochemistry in human keratinocytes. The cells, cultivated essentially as described above, were trypsinized, the solution centrifuged, and the pellet resuspended and transferred to a microscopic slide with cover slip. Immunocytochemistry was performed according to standard technique. BAX (N-20)-affinity purified rabbit polyclonal antibodies were purchased from Santa Cruz Biotechnology Inc. (Santa Cruz, Calif.), and used at a dilution of 1:200. Secondary goat-antirabbit antibodies conjugated to horseradish peroxidase (Dako Envision+TM) obtained from Dako (Carpinteria, Calif., USA) were used for detecting the primary antibodies. After appropriate rinsing the preparations were incubated with 2,2-diaminobenzidine as chromogenic substrate for the horseradish peroxidase for 10 min at room temperature. The endogenous peroxidases were quenched with hydrogen peroxide. Appropriate controls for immunocytochemical procedure were also included. The preparations were finally examined in a Nikon FTX fluorescence microscope equipped with camera, and colour photographs were taken of representative cells.

In another set of experiments, intravital staining for detection of apoptosis/necrosis in keratinocytes cultivated as above in the absence and presence of BR280 (17-phenyl-18,19,20-trinor-PGA₂) was performed with propidium-iodide/Hoechst 33342 (bisbenzimide) purchased from Sigma Chemical Company (St. Louis, Mo.). A mixture of propidium-iodide (10 μg/ml) and Hoechst 33342 (5 μg/ml) was added to the culture medium. The cells were incubated in the medium for 10 min at 37° C. The medium was harvested for free floating cells while the attached cells were washed once with Ca⁺⁺/Mg⁺⁺ free Hanks solution (Sigma Chemical Company), or PBS. Thereafter 1.5 ml of a 0.5% trypsin/EDTA solution (Ca⁺⁺/Mg⁺⁺ free Hanks solution, or PBS) was added and incubation was carried out for 5 min at 37° C. The solution was then centrifuged for 1 min (500 g). The pellet was resuspended in 0.5 ml PBS, and the solution was centrifuged as above. The pellet was then suspended in 20 μl PBS, and the solution containing the cells was placed on a microscopic slide with cover slip. The cells were examined in a Nikon FTX fluorescence microscope equipped with a UV-2A filter and camera. Colour photographs were taken of representative cells. The prostaglandin analogue (BR280) was diluted directly in the culture medium. The incubation time was 2 days in the immunocytochemical experiments (BAX), and 7 days in the experiments with intravital staining of the cells. In the longer experiments the culture medium containing BR280 was changed after 3-4 days of incubation.

BAX gene expression (BAX-protein) could be demonstrated in human keratinocytes exposed to BR280 at different concentrations whereas little or no immunostaining could be demonstrated in control keratinocyte cultures not exposed to the prostaglandin. At lower concentrations than 10⁻⁵ M the effect was weak or absent. Markedly condensed and fragmented nuclei and apoptotic bodies could be demonstrated by the intravital staining in the keratinocytes exposed for 7 days to BR280, typical for apoptosis. These experiments indicate that 17-phenyl-18,19,20-trinor-PGA₂ induces cell death through a mechanism of apoptosis in human keratinocytes in vitro.

17-phenyl-18,19,20-trinor-PGA₂ and functionally equivalent derivatives, and prodrugs thereof would thus be a promising alternative for the treatment and/or alleviation of psoriasis as they not only inhibit growth of keratinocytes, but also induce apoptosis, which is considered beneficial for the treatment.

Comparative Tests Performed During the Priority Year

17-phenyl-18,19,20-trinor-PGA₂ is Superior to PGA₂ with Respect to Side-Effect Profile.

PGA₂ and 17-phenyl-18,19,20-trinor-PGA₂, synthesized as described above, were formulated in vehicles suitable for hyperemia/irritation tests using the human eye as a model. The superficial redness of the eyes was documented by colour photographs before, and 15, 30, 60 and 120 min after application of the test substances. One eye was treated with the prostaglandin analogue and the other eye with the vehicle only. Ocular irritation or pain was recorded at the same time points. The two test substances at a concentration of 26 μg/ml (about 10⁻⁴ M) were tested in the same eye of three healthy individuals a few days apart.

About a week later, a higher concentration of each compound, 84 μg/ml of PGA₂ and 96 μg/ml of 17-phenyl-18,19,20-trinor-PGA₂ (both concentrations corresponding to about 3×10⁻⁴ M) was again tested a few days apart in the same eyes of the same individuals. The concentrations used are relevant since for the treatment of psoriasis the drugs have to be used at approximately this concentration range. The hyperemia was evaluated on a scale from 0 (no hyperemia) to 3 (maximum hyperemia) using half steps. A similar categorical scale was used for the ocular irritation or pain.

The inventors used the acids of the prostaglandins and not the ester prodrugs to demonstrate the inherent property of the pharmacologically active principle. In the skin, the prodrugs are hydrolysed to the corresponding acids, which then exert their pharmacological activity, both desirable and harmful. However, direct measurements of blood flow in the skin are difficult to perform. Hence, the inventors chose to use the eye as a suitable general model to study tissue hyperemia and irritation.

The results are presented in Table 2.

TABLE 2
Difference in ocular surface hyperemia and irritation or pain between
eyes treated with PGA₂ and 17-phenyl-18,19,20-trinor-PGA₂ (JB991 acid).
PGA₂ and JB991 acid at a concentration of 26 μg/ml were applied
topically on the same eye at separate occasions in 3 individuals. The same
procedure was repeated about one week later with higher concen-
tration (84 μg/ml) (17-phenyl-18,19,20-trinor-PGA₂ was in fact tested at a
dose of 96 μg/ml). In none of the individuals was any hyperemia or
ocular irritation or pain noted after application of JB991 acid. (Mean ±
SEM; n = 3).

| Time after | | | | |
|---|---|---|---|---|
| adminis- | Difference in | | Difference in | |
| tration | hyperemia | | irritation | |
| of test | PGA₂ - JB991 acid | | PGA₂ - JB991 acid | |
| substances | Dose 1 | Dose 2 | Dose 1 | Dose 2 |
| (min) | (26 μg/ml) | (84 μg/ml) | (26 μg/ml) | (84 μg/ml) |
| 0 | 0.00 ± 0.00 | 0.00 ± 0.00 | 0.00 ± 0.00 | 0.00 ± 0.00 |
| 15 | 1.33 ± 0.60 | 2.17 ± 0.44 **) | 1.00 ± 0.29 | 1.83 ± 0.44 *) |
| 30 | 1.17 ± 0.44 | 2.17 ± 0.44 **) | 1.17 ± 0.33 | 1.33 ± 0.60 |
| 60 | 0.67 ± 0.44 | 1.33 ± 0.60 | 0.50 ± 0.29 | 0.83 ± 0.44 |
| 120 | 0.17 ± 0.17 | 0.50 ± 0.29 | 0.17 ± 0.17 | 0.33 ± 0.17 |

*) p < 0.02;
**) p < 0.01 with Student's t-test between group means.

In conclusion, 17-phenyl-18,19,20-trinor-PGA₂ exhibits exceptional, novel properties as a remedy for psoriasis because:
1. The prostaglandin analogue was found to exert excellent antiproliferative effect on human keratinocytes, even better than PGA₂,
2. In contrast to PGA₂ it was completely devoid of any side-effects, whatsoever, when tested in a sensitive hyperemia/irritation model,
3. Among the omega chain ring-substituted analogues tested it exhibited by far the best antiproliferative effect on human keratinocytes.
4. 17-phenyl-18,19,20-trinor PGA₂ has been found to exert marked apoptotic effect on human keratinocytes, an important mechanism to prevent the thickening of the skin in the psoriatic skin plaques.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

1-5. (canceled)

6. A composition for topical application, said composition containing a therapeutically active and physiologically acceptable amount of 17-phenyl-18,19,20-trinor-PGA₂ or a derivative thereof for the treatment and/or alleviation of psoriasis.

7. The composition according to claim 6, wherein the prostaglandin is an ester prodrug.

8. The composition according to claim 6, wherein the prostaglandin is an amide prodrug.

9. The composition according to claim 7, wherein the prostaglandin is 17-phenyl-18,19,20-trinor-PGA₂-isopropyl ester.

10. The composition according to claim 6, wherein the composition can be applied topically in a therapeutically active amount without or with only minimal side-effects, such as hyperemia, irritation or pain.

11. A method for the treatment and/or alleviation of psoriasis in a human patient, wherein a therapeutically active and physiologically acceptable amount of a prostaglandin is applied topically to a psoriatic lesion on the skin of said patient, wherein said prostaglandin is 17-phenyl-18,19,20-trinor-PGA₂ or a derivative or prodrug thereof.

12. The method according to claim 11, wherein the prostaglandin is an ester prodrug.

13. The method according to claim 11, wherein the prostaglandin is an amide prodrug.

14. The method according to claim 12, wherein the prostaglandin is 17-phenyl-18,19,20-trinor-PGA₂-isopropyl ester.

15. A method according to claim 11, wherein the treatment and/or alleviation of psoriasis can be performed without or with only minimal side-effects, such as hyperemia, irritation or pain.
